(19) United States
(12) Patent Application Publication
(10) Pub. No.: US 2014/0093468 A1
(43) Pub. Date: Apr. 3, 2014

(54) STYLING AGENTS WITH AN INTERESTING TEXTURE

(71) Applicant: Henkel AG & Co. KGaA, Duesseldorf (DE)

(72) Inventors: Thorsten Knappe, Schenefeld (DE); Marcus Noll, Norderstedt (DE)

(73) Assignee: Henkel AG & Co. KGaA, Duesseldorf (DE)

(21) Appl. No.: 14/100,151

(22) Filed: Dec. 9, 2013

Related U.S. Application Data
(63) Continuation of application No. PCT/EP2012/060718, filed on Jun. 6, 2012.

(30) Foreign Application Priority Data
Jun. 10, 2011 (DE) 10 2011 077 365.7

(51) Int. Cl. A61K 8/60; A61K 8/04; A61Q 5/06; A61K 8/81; A61K 8/92

(52) U.S. Cl. CPC A61K 8/604; A61K 8/8176; A61K 8/925; A61Q 5/06; A61K 8/046
USPC 424/70.15

(57) ABSTRACT

Agents for the temporary shaping of keratinic fibers are characterized by a pleasant product feel, light consistency and a lightweight application. The agents include in a cosmetically acceptable carrier, relative in each case to the total agent, 0.1 to 15 wt. % of monoesters of optionally alkylated sugars with C₆-C₃₀ fatty acids, 0.1 to 15 wt. % of diesters of optionally alkylated sugars with C₆-C₃₀ fatty acids, 0 to 50 wt. % of at least one film-forming polymer, 0 to 50 wt. % of at least one wax with a melting point in a range from 40° C. to 90° C., with the proviso that the proportion of component(s) c) and d) in the cosmetic agent is 0.2 to 50 wt. %.

### FIELD OF THE INVENTION

The present invention generally relates to agents for the temporary shaping of keratinic fibers, containing a combination of at least one monoester and at least one diester of optionally alkylated sugars with C₆-C₃₀ fatty acids and further special ingredients, to the use of said agents for the temporary shaping of keratinic fibers and to a corresponding method.

### BACKGROUND OF THE INVENTION

Keratinic fibers in principle include all animal hair, for example wool, horsehair, angora hair, fur, feathers and products or textiles manufactured therefrom. The keratinic fibers are however preferably human hair.

An attractive-looking hairstyle is today regarded as an essential element of a well-groomed exterior. The latest fashion trends mean that for many hair types, hairstyles that are regarded as fashionable can only be constructed or maintained for an extended period of up to several days using active fixing agents. Therefore hair treatment agents that provide a permanent or temporary shaping of the hair have an important role to play. Temporary shaping effects which offer good hold without adversely affecting the healthy appearance of the hair, such as its shine for example, can be achieved for example by means of hair sprays, hair waxes, hair gels, hair foams, blow-drying lotions, etc.

Corresponding agents for temporary shaping conventionally contain synthetic polymers as the shaping component. Preparations containing a dissolved or dispersed polymer can be applied to the hair by means of propellants or via a pump mechanism. Hair gels and hair waxes in particular however are generally not applied directly to the hair but are distributed in the hair using a comb or the hands.

The most important property of an agent for temporarily shaping keratinic fibers, also referred to below as a styling agent, consists in giving the treated fibers the strongest possible hold in the created shape. If the keratinic fibers are human hair, this property is also described as a strong styling hold or a high degree of hold of the styling agent. The styling hold is substantially determined by the nature and amount of the synthetic polymer that is used, although the further constituents of the styling agent can also have an influence.

There is a problem in that consumers have a negative view of the consistency of such products, since they are considered to be viscous, sticky and difficult to apply and they leave an oily, heavy feel on the hair. In addition, consumers would like products that have a pleasant feel and texture and are perceived to be “different.”

It is therefore desirable to provide an agent for temporarily shaping keratinic fibers that is distinguished by a pleasant product feel, a light consistency and a lightweight application.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic agent for the temporary shaping of keratinic fibers, containing in a cosmetically acceptable carrier, relative in each case to the total agent, 0.1 to 15 wt. % of monoesters of optionally alkylated sugars with C₆-C₃₀ fatty acids; 0.1 to 15 wt. % of diesters of optionally alkylated sugars with C₆-C₃₀ fatty acids; 0 to 50 wt. % of at least one film-forming polymer; 0 to 50 wt. % of at least one wax with a melting point in a range from 40° C. to 90° C.; with the proviso that the proportion of component(s) c) and d) in the cosmetic agent is 0.2 to 50 wt. %.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly it has been found that lightweight styling products having a high degree of hold and a pleasant conditioning feel in the hair can be provided by incorporating a combination of special monoesters and diesters of optionally alkylated sugars with C₆-C₃₀ fatty acids and further special ingredients into the agents.

The present invention therefore firstly provides a cosmetic agent containing in a cosmetically acceptable carrier, relative in each case to the total agent,
a) 0.1 to 15 wt. % of monoesters of optionally alkylated sugars with C₆-C₃₀ fatty acids,
b) 0.1 to 15 wt. % of diesters of optionally alkylated sugars with C₆-C₃₀ fatty acids,
c) 0 to 50 wt. % of at least one film-forming polymer,
d) 0 to 50 wt. % of at least one wax with a melting point in a range from 40° C. to 90° C.,
   with the proviso that the proportion of component(s) c) and d) in the cosmetic agent is 0.2 to 50 wt. %.

The agents according to the invention contain as the first required component 0.1 to 15 wt. % of monoesters of optionally alkylated sugars with C₆-C₃₀ fatty acids. Monosaccharides, disaccharides and/or oligosaccharides as well as the alkyl-substituted derivatives thereof can be used to particular advantage as the sugar building block in said monoesters. More preferred monosaccharides are in particular D-ribose and/or D-xylose and/or L-arabinose and/or D-glucose and/or D-mannose and/or D-galactose and/or D-fructose and/or sorbose and/or L-fucose and/or L-rhamnose, while more preferred disaccharides are in particular sucrose and/or maltose and/or lactose and/or trehalose and/or cellobiose and/or gentiobiose and/or isomaltose.

Alkylated sugars can also be used as the sugar building block of monoesters of sugars with C₆-C₃₀ fatty acids. Alkyl glycosides, alkyl oligoglycosides and alkyl polyglycosides have proved their worth here in particular. Alkyl polyglycosides corresponding to the general formula RO—(Z)ₓ in which R denotes alkyl, Z denotes sugar and x denotes the number of sugar units, are more preferred here. The alkyl polyglycosides for use according to the invention can only contain one particular alkyl residue R. These compounds are conventionally produced from natural fats and oils or mineral oils, however. In this case mixtures corresponding to the starting compounds or to the processing of said compounds are present as alkyl residues R.

Alkyl polyglycosides in which R consists
substantially of C₈ and C₁₀ alkyl groups
substantially of C₁₂ and C₁₄ alkyl groups
substantially of C₈ to C₁₆ alkyl groups or substantially of C₁₂ to C₁₆ alkyl groups or
substantially of C₁₆ to C₁₈ alkyl groups
   are more preferred.

Any mono- or oligosaccharides can be used in turn as the sugar building block Z. Sugars having 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is more preferred.

The alkyl polyglycosides used according to the invention as the sugar building block contain on average 1.1 to 5 sugar units. Alkyl polyglycosides having x values from 1.1 to 2.0 are preferred. Alkyl glycosides in which x is 1.1 to 1.8 are particularly preferred.

All C₆-C₃₀ fatty acids can be used as the fatty acid building block of the monoesters of optionally alkylated sugars with C₆-C₃₀ fatty acids, with the saturated acids hexanoic acid, heptanoic acid, octanoic acid, pelargonic acid, decanoic acid, lauric acid, myristic acid, palmitic acid, margaric acid, stearic acid, eicosanoic acid, behenic acid, lignoceric acid, cerotic acid, montanic acid and melissic acid being more preferred.

Unsaturated fatty acids can have a double bond in the carbon chain in both the (E−) and the (Z−) configuration, wherein if there is a plurality of double bonds in the fatty acid, fatty acids with double bonds exclusively in the (Z−) configuration, exclusively in the (E−) configuration or in mixed configuration can be used. Unsaturated fatty acids having an all-(Z−) configuration are preferred, however. Unsaturated fatty acids that are preferred according to the invention are palmitoleic acid (C16:1; 9Z), oleic acid (C18:1; 9Z), elaidic acid (C18:1; 9E), eicosenic acid (gondoic acid; C20:1; 11Z), erucic acid (C22:1; 13Z), nervonic acid (C24:1; 15Z), linoleic acid (C18:2; 9Z, 12Z), γ-(gamma)-linolenic acid (C18:3; 6Z, 9Z, 12Z), α-(alpha)-linolenic acid (C18:3; 9Z, 12Z, 15Z), α-elaeostearic acid (C18:3; 9Z, 11E, 13E) and arachidonic acid (C20:4; 5Z, 8Z, 11Z, 14Z).

In particularly preferred agents according to the invention component a) is selected from sucrose monostearate, sucrose monococoate, methyl glucoside monostearate, ethyl glucoside monolaurate, ethyl glucoside monococoate and butyl glucoside monococoate.

Irrespective of whether one monoester or a mixture of various monoesters is used, cosmetic agents according to the invention which contain 0.2 to 12 wt. %, preferably 0.3 to 10 wt. %, more preferably 0.4 to 5 wt % and in particular 0.5 to 3 wt. % of monoesters of optionally alkylated sugars with C₆-C₃₀ fatty acids are preferred.

As the second essential component the agents according to the invention contain 0.1 to 15 wt. % of diesters of optionally alkylated sugars with C₆-C₃₀ fatty acids. Both sugar building blocks and fatty acid building blocks can come from the aforementioned groups of monoesters. It is preferable for the diester not to be a mixed diester but rather for both residues to come from the same fatty acid. In particularly preferred cosmetic agents according to the invention component b) is selected from sucrose distearate, sucrose dicocoate, methyl glucoside isostearate, ethyl glucoside dilaurate and ethyl glucoside dicocoate.

Irrespective of whether one diester or a mixture of various diesters is used, cosmetic agents according to the invention which contain 0.2 to 12 wt. %, preferably 0.3 to 10 wt. %, more preferably 0.4 to 5 wt. % and in particular 0.5 to 3 wt. % of diesters of optionally alkylated sugars with C₆-C₃₀ fatty acids are preferred.

A particularly preferred monoester according to the invention is sucrose monostearate, while a particularly preferred diester according to the invention is sucrose distearate. Said two compounds are particularly preferably used together, wherein more preferred cosmetic agents according to the invention contain 0.25 to 5 wt. %, preferably 0.5 to 4 wt. %, more preferably 0.6 to 3 wt. % and in particular 0.75 to 1.5 wt. % of sucrose monostearate and 0.25 to 5 wt. %, preferably 0.5 to 4 wt. %, more preferably 0.6 to 3 wt. % and in particular 0.75 to 1.5 wt. % of sucrose distearate.

As a further essential component the agents according to the invention contain either 0 to 50 wt. % of at least one film-forming polymer or 0 to 50 wt. % of at least one wax having a melting point in a range from 40° C. to 90° C. or a mixture of compound(s) of both classes of substances, with the proviso that the proportion of component(s) c) and d) in the cosmetic agent is 0.2 to 50 wt. %.

Components c) and d) are described below. The agents according to the invention can contain 0 to 50 wt. % of at least one film-forming polymer. They can of course also contain a plurality of film-forming and/or fixing polymers. These film-forming and/or fixing polymers can be both permanently and temporarily cationic, anionic, non-ionic or amphoteric. Where at least two film-forming and/or fixing polymers are used, they can naturally have differing charges. It can be preferred according to the invention for an ionic film-forming and/or fixing polymer to be used together with an amphoteric and/or non-ionic film-forming and/or fixing polymer. The use of at least two oppositely charged film-forming and/or fixing polymers is also preferred. In the latter case a particular embodiment can in turn additionally contain at least one further amphoteric and/or non-ionic film-forming and/or fixing polymer.

As polymers are commonly multifunctional, their functions cannot always be clearly and unambiguously separated from one another. This is particularly true of film-forming and fixing polymers.

Many polymers that are primarily described as film-forming also have fixing properties, and vice versa. Therefore it is explicitly stated at this point that in the context of the present invention both film-forming and fixing polymers are essential. As the two properties are not entirely independent of one another, the term “fixing polymers” is always understood also to mean “film-forming polymers” and vice versa.

One of the preferred properties of film-forming polymers is film formation. Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair masks, shampoos or nail varnishes. Polymers having an adequate solubility in water, alcohol or water/alcohol mixtures are preferred in particular. Thus, corresponding solutions that are simple to use or to process further can be produced. The film-forming polymers can be of synthetic or natural origin. Film-forming polymers are also understood to be polymers which when used in a 0.01 to 20 wt. % aqueous, alcoholic or aqueous-alcoholic solution are capable of depositing a transparent polymer film on the hair. The film-forming polymers can be anionically, amphoterically, non-ionically, permanently cationically or temporarily cationically charged.

Suitable synthetic, film-forming, hair-fixing polymers that are preferably used according to the invention are homopolymers or copolymers synthesized from at least one of the following monomers: vinylpyrrolidone, vinylcaprolactam, vinyl esters such as for example vinyl acetate, vinyl alcohol, acrylamide, methacrylamide, C₁ to C₇ alkyl acrylamide, C₁ to C₇ dialkyl acrylamide, C₁ to C₇ alkyl methacrylamide, C₁ to C₇ dialkyl methacrylamide, C₁ to C₇ alkyl acrylate, acrylic acid, propylene glycol, ethylene glycol, the C₁ to C₇ alkyl groups of said monomers preferably being C₁ to C₃ alkyl groups.

Homopolymers of vinylcaprolactam, vinylpyrrolidone or N-vinylformamide are cited by way of example. Further suitable synthetic film-forming, hair-fixing polymers are for example copolymers of vinylpyrrolidone and vinyl acetate, terpolymers of vinylpyrrolidone, vinyl acetate and vinyl propionate, polyacrylamides, which are sold for example under the trade names Akypomine® P 191 by CHEM-Y, Emmerich, or Sepigel® 305 by Seppic; polyvinyl alcohols, which are sold for example under the trade names Elvanol® by Du Pont or Vinol® 523/540 by Air Products, and polyethylene glycol/polypropylene glycol copolymers, which are sold for example under the trade names Ucon® by Union Carbide.

Suitable natural film-forming polymers are for example cellulose derivatives, for example hydroxypropyl cellulose having a molecular weight from 30,000 to 50,000 g/mol, which are sold for example under the trade name Nisso Sl® by Lehmann & Voss, Hamburg.

Fixing polymers contribute to maintaining and/or establishing volume and fullness in the hairstyle as a whole. These so-called fixing polymers are simultaneously also film-forming polymers and are therefore generally typical substances for hair treatment agents used for shaping, such as hair fixing agents, hair foams, hair waxes, hair sprays. Film formation may be entirely localized here and may bond only a few fibers together.

Substances which also impart hydrophobic properties to the hair are preferred here because they reduce the tendency of the hair to absorb moisture, i.e. water. This prevents strands of hair from drooping and thus ensures lasting shape and hold for the hairstyle. The curl retention test is often used as a test method here. These polymeric substances can furthermore be incorporated into leave-on and rinse-off hair masks or shampoos. As polymers are frequently multifunctional, i.e. they demonstrate multiple effects which are desirable from an applications perspective, many polymers are categorized into more than one group classified by mode of action, as is the case in the CTFA handbook. Owing to the significance of the fixing polymers, they should therefore be listed explicitly in the form of their INCI names. Therefore this list naturally also includes the cited film-forming polymers.

Examples of common film-forming, fixing polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacryl ate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/lsophthalic Acid/Neopentyl Glycol/Trimethylolpropane Copolymer, Allyl Stearate/VA Copolymer, Amino ethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquatemium-1, Polyquaternium-2, Polyquatemium-4, Polyquaternium-5, Polyquatemium-6, Polyquaternium-7, Polyquatemium-8, Polyquatemium-9, Polyquatemium-10, Polyquatemium-11, Polyquatemium-12, Polyquatemium-13, Polyquatemium-14, Polyquatemium-15, Polyquatemium-16, Polyquatemium-17, Polyquatemium-18, Polyquatemium-19, Polyquatemium-20, Polyquaternium-22, Polyquatemium-24, Polyquaternium-27, Polyquaternium-28, Polyquaternium-29, Polyquatemium-30, Polyquatemium-31, Polyquatemium-32, Polyquaternium-33, Polyquaternium-34, Polyquatemium-35, Polyquaternium-36, Polyquatemium-37, Polyquatemium-39, Polyquaternium-45, Polyquaternium-46, Polyquaternium-47, Polyquaternium-48, Polyquaternium-49, Polyquatemium-50, Polyquaternium-55, Polyquatemium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/lsophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate.

Compositions according to the invention preferably contain at least one film-forming and/or fixing polymer selected from vinylpyrrolidone/vinyl acetate copolymers, vinyl acetate/crotonic acid copolymers, vinylcaprolactam/vinylpyrrolidone/dimethylaminoethyl methacrylate copolymers, octyl acrylamide/acrylate/butylaminoethyl methacrylate copolymers and quaternized vinylpyrrolidone/dimethylaminoethyl methacrylate copolymers.

The film-forming and/or fixing polymer is more preferably the vinylpyrrolidone/vinyl acetate copolymer Luviskol® VA 37 or PVP/VA copolymer 60/40 W NP, the vinyl acetate/crotonic acid copolymer sold under the trade name Aristoflex® A 60, the vinylcaprolactam/vinylpyrrolidone/dimethylaminoethyl methacrylate copolymer with the trade name Advantage® LC-E, the amphoteric octylacrylamide/acrylates/butylaminoethyl methacrylate copolymer available under the name Amphomer® or the vinylpyrrolidone/dimethylaminoethyl methacrylate copolymer quaternized by reaction with diethyl sulfate, which is sold under the trade name Gafquat® 755N.

In particular, compositions according to the invention preferably additionally contain at least one vinylpyrrolidone/vinyl acetate copolymer.

In summary, agents according to the invention are preferred in which the film-forming polymer and/or polymer having a fixing action on the hair is selected from at least one polymer from the group formed by non-ionic polymers based on ethylenically unsaturated monomers, non-ionic cellulose derivatives, starch and derivatives thereof, chitosan and derivatives of chitosan, cationic cellulose derivatives, cationic copolymers of 3-(C₁ to C₆)-alkyl-1-vinyl imidazolinium, homopolymers and copolymers of diallyl dimethyl ammonium, homopolymers and copolymers containing the structural unit of formula (M−1)

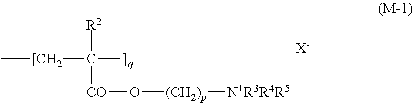

in which R² is —H or —CH₃, R³, R⁴ and R⁵ are selected independently of one another from (C₁ to C₄) alkyl, (C₁ to C₄) alkenyl or (C₂ to C₄) hydroxyalkyl groups, p=1, 2, 3 or 4, q is a natural number and X⁻ is a physiologically acceptable organic or inorganic anion, amphoteric copolymers of N—(C₆ to Cₘ) alkyl acrylamide, amphoteric copolymers of N—(C₆ to C₁₀) alkyl methacrylamide, anionic polymers containing carboxylate and/or sulfonate groups, anionic polyurethanes.

In summary, agents according to the invention containing 0.2 to 15 wt. %, preferably 0.4 to 10 wt. %, more preferably 0.6 to 7 wt. % and in particular 0.75 to 5 wt. % of film-forming polymer(s) are preferred.

In addition to the film-forming polymer(s) or in place thereof, the agents according to the invention can contain 0 to 50 wt. % of at least one wax having a melting point in a range from 40° C. to 90° C.

Waxes are generally of a solid to crumbly hard consistency, coarsely to finely crystalline, translucent to opaque, but not glassy, and melt above 40° C. without decomposing. At even a little above the melting point they are of low viscosity and have a highly temperature-dependent consistency and solubility. Natural vegetable waxes, for example candelilla wax, carnauba wax, Japan wax, sugar cane wax, ouricury wax, cork wax, sunflower wax, fruit waxes such as orange wax, lemon wax, grapefruit wax, and animal waxes, for example beeswax, shellac wax and spermaceti wax, are preferred for example according to the invention. Within the meaning of the invention it can be more preferable to use hydrogenated or hardened waxes. Chemically modified waxes, in particular hard waxes such as for example montan ester waxes, hydrogenated jojoba waxes and Sasol waxes, can also be used as the wax component. Synthetic waxes that are likewise preferred according to the invention include for example polyalkylene waxes, in particular polyethylene waxes, and polyethylene glycol waxes, C₂₀-C₄₀ dialkyl esters of dimeric acids, C₃₀₋₅₀ alkyl beeswax and alkyl and alkylaryl esters of dimeric fatty acids.

A more preferred wax component is selected from at least one ester of a saturated, monohydric C₁₆-C₅₀ alcohol and a saturated C₈-C₃₆ monocarboxylic acid. According to the invention these also include lactides, the cyclic double esters of alpha-hydroxycarboxylic acids of the corresponding chain length. Esters of fatty acids and long-chain alcohols have proved particularly advantageous for the compositions that are preferred according to the invention. The esters consist of saturated, branched or unbranched monocarboxylic acids and saturated, branched or unbranched monohydric alcohols. Esters of aromatic carboxylic acids or hydroxycarboxylic acids (e.g. 12-hydroxystearic acid) and saturated, branched or unbranched alcohols can also be used according to the invention, provided that the wax component has a melting point>50° C. It is more preferable to select the wax components from the group of esters of saturated, branched or unbranched alkane carboxylic acids having a chain length of 12 to 24 C atoms and saturated, branched or unbranched alcohols having a chain length of 16 to 50 C atoms that have a melting point of >50° C.

In particular, C₁₆₋₃₆ alkyl stearates and C₁₈₋₃₈ alkylhydroxystearoyl stearates, C₂₀₋₄₀ alkyl erucates and cetearyl behenate can be preferred as the wax component. The wax or wax components have a melting point>50° C., preferably >60° C.

A more preferred embodiment of the invention contains a C₂₀-C₄₀ alkyl stearate as the wax component. This ester is known under the name Kesterwachs® K82H or Kesterwachs® K80H and is sold by Koster Keunen Inc. It is the synthetic simulation of the monoester fraction of beeswax and is distinguished by its hardness, its oil gelling capacity and its broad compatibility with lipid components. A further more preferred embodiment of the invention contains cetearyl behenate, i.e. mixtures of cetyl behenate and stearyl behenate, as the wax component. This ester is known under the name Kesterwachs® K62 and is sold by Koster Keunen Inc.

Other preferred wax components having a melting point>50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, also synthetic full esters of fatty acids and glycols or polyols having 2 to 6 carbon atoms, provided they have a melting point above 50° C., for example preferably C₁₈-C₃₈ acid triglyceride (Syncrowax® HGL-C). Hydrogenated castor oil, obtainable for example as the commercial product Cutina® HR, is more preferred according to the invention as the wax component.

Further preferred lipid or wax components having a melting point>50° C. are the saturated linear C₁₄-C₃₆ carboxylic acids, in particular myristic acid, palmitic acid, stearic acid and behenic acid, as well as mixtures of these compounds, for example Syncrowax® AW IC (C₁₈-C₃₆ fatty acids) or Cutina® FS 45 (palmitic and stearic acid).

Further preferred lipid or wax components having a melting point in the range from 30 to 150° C. are linear, saturated C₈₋₃₀ fatty acids. Linear, saturated C₁₀₋₂₂ fatty acids are preferred. Preferred fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, elaeostearic acid, arachidonic acid, gadoleic acid, behenic acid and erucic acid and technical mixtures thereof. The use of stearic acid is more preferred. The fatty acids that are used can bear one or more hydroxyl groups. Preferred examples are α-hydroxy-C₈-C₁₈-carboxylic acids and 12-hydroxystearic acid.

Further preferred lipid or wax components having a melting point in the range from 30 to 150° C. are fatty alcohols. Saturated, unbranched fatty alcohols having 6-30, preferably 10-22 and particularly preferably 12-22 carbon atoms can be used as fatty alcohols. Decanol, octanol, erucic alcohol, ricinol alcohol, 12-hydroxystearyl alcohol, stearyl alcohol, cetyl alcohol, lauryl alcohol, myristyl alcohol, arachidyl alcohol, capryl alcohol, caprinic alcohol and behenyl alcohol, for example, can be used within the meaning of the invention.

Preferred compositions according to the invention are characterized in that the wax component is selected from esters of a saturated, monohydric C₁₆-C₆₀ alkanol and a saturated C₆-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which can be hydroxylated, candelilla wax, carnauba wax, beeswax, saturated linear C₁₄-C₃₆ carboxylic acids and mixtures of the aforementioned substances. More preferred wax component mixtures are selected from mixtures of cetyl behenate, stearyl behenate, hydrogenated castor oil, palmitic acid and stearic acid. Further more preferred wax component mixtures are selected from mixtures of C₂₀-C₄₀ alkyl stearate, hydrogenated castor oil, palmitic acid and stearic acid.

More preferred compositions according to the invention are characterized in that the wax component is selected from mixtures of esters of a saturated, monohydric C₁₆-C₆₀ alkanol and a saturated C₈-C₃₆ monocarboxylic acid, in particular C₂₀-C₄₀ alkyl stearate, glycerol triesters of saturated linear C₁₂-C₃₀ carboxylic acids, which can be hydroxylated, in particular hydrogenated castor oil, and saturated linear C₁₄-C₃₆ carboxylic acids, in particular palmitic acid and stearic acid.

In summary, agents that are more preferred according to the invention are characterized in that they contain 1 to 45 wt. %, preferably 2.5 to 40 wt. %, more preferably 3 to 30 wt. % and in particular 45 to 25 wt. % of at least one wax from the group comprising beeswax, carnauba wax, candelilla wax, montan wax and/or cetyl palmitate.

It has proved particularly advantageous for the shaping effect for the weight ratio of the wax contained in the agent to the esters of optionally alkylated sugars with C₆-C₃₀ fatty acids contained in the agent to be >8:1, preferably 8:1 to 30:1 and in particular 10:1 to 20:1. Corresponding agents are preferred according to the invention.

As already mentioned, the agents can contain either film-forming polymer(s) or wax(es) or a mixture of substances of both substance classes, with the proviso that the proportion of component(s) c) and d) in the cosmetic agent is 0.2 to 50 wt. %. The joint use of both components is more preferred, i.e. preferred agents contain both at least one film-forming polymer and at least one wax.

More preferred agents are characterized in that they contain, relative to their weight, 1 to 45 wt. %, preferably 2.5 to 40 wt. %, more preferably 5 to 30 wt. % and in particular 7.5 to 25 wt. % of at least one wax from the group comprising beeswax, carnauba wax, candelilla wax, montan wax and/or cetyl palmitate and 0.2 to 15 wt. %, preferably 0.4 to 10 wt. %, more preferably 0.6 to 7 wt. % and in particular 0.75 to 5 wt. % of film-forming polymer(s). More preferred cosmetic agents are characterized in that the weight ratio of the wax contained in the agent to the film-forming polymer contained in the agent is >8:1, preferably between 8:1 and 30: and in particular between 10:1 and 20:1.

Particularly preferred agents are characterized in that they contain, relative to their weight, 1 to 45 wt. %, preferably 2.5 to 40 wt. %, more preferably 5 to 30 wt. % and in particular 7.5 to 25 wt. % of beeswax and 0.2 to 15 wt. %, preferably 0.4 to 10 wt. %, more preferably 0.6 to 7 wt. % and in particular 0.75 to 5 wt. % of film-forming polymer(s).

The agents according to the invention contain the ingredients in a cosmetically acceptable carrier. Preferred cosmetically acceptable carriers are aqueous, alcoholic or aqueous-alcoholic media having preferably at least 10 wt. % water, relative to the total agent. The low alcohols having 1 to 4 carbon atoms that are conventionally used for cosmetic purposes, such as for example ethanol and isopropanol, can be included in particular as alcohols.

Organic solvents or a mixture of solvents with a boiling point below 400° C. can be included as additional co-solvents in an amount from 0.1 to 15 percent by weight, preferably from 1 to 10 percent by weight, relative to the total agent. Unbranched or branched hydrocarbons, such as pentane, hexane, isopentane, and cyclic hydrocarbons, such as cyclopentane and cyclohexane, are particularly suitable as additional co-solvents. Other more preferred water-soluble solvents are glycerol, ethylene glycol, butylene glycol and propylene glycol in an amount of up to 30 wt. % relative to the total agent.

Particularly preferred agents according to the invention have a high water content. It has been found that the adjustment of shine, remodeling ability and degree of hold in the compositions according to the invention is particularly successful if they contain high proportions of water. More preferred agents according to the invention are therefore characterized in that they contain, relative to their weight, 40 to 95 wt. %, preferably 45 to 92.5 wt. %, more preferably 50 to 90 wt. %, still more preferably 55 to 87.5 wt. % and in particular 60 to 85 wt. % of water. Preferred agents according to the invention are in the form of an oil-in-water emulsion.

The applicability of the compositions according to the invention can be further increased through the use of small amounts of one or more polyhydric alcohols. Preferred agents according to the invention contain, relative to their weight, 0.25 to 5 wt. %, preferably 0.5 to 4 wt. %, more preferably 0.75 to 3 wt. % and in particular 1 to 2.5 wt. % of at least one polyhydric alcohol form the group comprising glycerol and/or propanediol-1,2.

The agents preferably have a pH of 2 to 11. The pH range between 4 and 9 is more preferred. Unless otherwise specified, within the meaning of this document the pH values stated relate to the pH at 25° C.

The agents according to the invention can furthermore contain the auxiliary substances and additives that are conventionally added to the various cosmetic agents.

Suitable auxiliary substances and additives include in particular conditioning substances. These are used in both skin and hair treatment agents and with an appropriate choice of conditioning substance can be incorporated into creams, shampoos, hair rinses, hair masks, gels, pump and aerosol sprays and foam products, for example.

An agent according to the invention can for example contain at least one protein hydrolysate and/or one of the derivatives thereof as a conditioning substance.

Protein hydrolysates are mixtures of products which are obtained by acidically, basically or enzymatically catalyzed breakdown of proteins. The molecular weight of the protein hydrolysates for use according to the invention is between 75, the molecular weight for glycine, and 200,000; the molecular weight is preferably 75 to 50,000 and particularly preferably 75 to 20,000 daltons. According to the invention protein hydrolysates of both plant and animal or marine or synthetic origin can be used. Animal protein hydrolysates are for example elastin, collagen, keratin, silk and milk protein hydrolysates, which can also be present in the form of salts. The use of silk protein hydrolysates is of particular interest. The protein hydrolysates are contained in the agents according to the invention in concentrations for example from 0.01 wt. % to 20 wt. %, preferably from 0.05 wt. % to 15 wt. % and particularly preferably in amounts from 0.05 wt. % to 5 wt. %, relative in each case to the total application preparation.

Cationic surfactants are moreover suitable as a conditioning substance from a different class of compounds. Cationic surfactants of the quaternary ammonium compound, esterquat and amidoamine type are preferred according to the invention. Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI names Quaternium-27 and Quaternium-83. The long alkyl chains of the aforementioned surfactants preferably have 10 to 18 carbon atoms. Esterquats are known substances containing both at least one ester function and at least one quaternary ammonium group as a structural element. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines. Such products are sold under the trademarks Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU-35 are examples of such esterquats. The alkylamidoamines are conventionally produced by amidation of natural or synthetic fatty acids and fatty acid cuts with dialkyl amino amines. A compound from this group of substances that is particularly suitable according to the invention is the stearamidopropyl dimethylamine which is commercially available under the name Tegoamid® S 18.

The cationic surfactants are preferably contained in the agents according to the invention in amounts from 0.05 to 10 wt. %, relative to the total application preparation. Amounts from 0.1 to 5 wt. % are more preferred.

Conditioning polymers are likewise suitable as a conditioning substance. It should be mentioned at this point that some conditioning polymers also have film-forming and/or fixing properties and can therefore also be included in the list of suitable film-forming and/or fixing polymers.

A first group of conditioning polymers are the cationic polymers. Cationic polymers are understood to be polymers having a group in the main and/or side chain which can be “temporarily” or “permanently” cationic. Polymers which have a cationic group irrespective of the pH of the agent are described as “permanently cationic” according to the invention. These are generally polymers containing a quaternary nitrogen atom, in the form of an ammonium group for example. Preferred cationic groups are quaternary ammonium groups. In particular, polymers in which the quaternary ammonium group is bound via a C₁₋₄ hydrocarbon group to a polymer main chain synthesized from acrylic acid, methacrylic acid or derivatives thereof have proved to be particularly suitable.

Homopolymers of the general formula (G1-I),

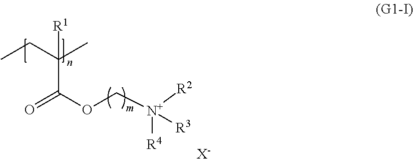

in which R¹ is —H or —CH₃, R², R³ and R⁴ are independently of one another selected from C₁₋₄ alkyl, alkenyl or hydroxyalkyl groups, m=1, 2, 3 or 4, n is a natural number and X⁻ is a physiologically acceptable organic or inorganic anion, as well as copolymers consisting substantially of the monomer units shown in formula (G1-I) along with non-ionogenic monomer units, are more preferred cationic polymers. In the context of these polymers, those for which at least one of the following conditions applies are preferred according to the invention: R¹ denotes a methyl group, R², R³ and R⁴ denote methyl groups, m has the value 2.

Suitable physiologically acceptable counterions X⁻ are for example halide ions, sulfate ions, phosphate ions, methosulfate ions as well as organic ions such as lactate, citrate, tartrate and acetate ions. Halide ions, in particular chloride, are preferred.

A particularly suitable homopolymer is the optionally crosslinked poly(methacryloyloxyethyl trimethylammonium chloride) with the INCI name Polyquaternium-37. Crosslinking can take place if desired with the aid of polyolefinically unsaturated compounds, for example divinyl benzene, tetraallyl oxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably used in the form of a non-aqueous polymer dispersion which should have a polymer content of not less than 30 wt. %. Such polymer dispersions are commercially available under the names Salcare® SC 95 (approx. 50% polymer content, other components: mineral oil (INCI name: Mineral Oil) and tridecyl polyoxypropylene polyoxyethylene ether (INCI name: PPG-1-Trideceth-6)) and Salcare® SC 96 (approx. 50% polymer content, other components: mixture of diesters of propylene glycol with a mixture of octanoic and decanoic acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (G1-I) preferably contain as non-ionogenic monomer units acrylamide, methacrylamide, acrylic acid C₁₋₄ alkyl esters and methacrylic acid C₁₋₄ alkyl esters. Of these non-ionogenic monomers acrylamide is more preferred. As in the case of the homopolymers described above, these copolymers too can be crosslinked. A copolymer that is preferred according to the invention is the crosslinked acrylamide-methacryloyloxyethyl trimethylammonium chloride copolymer. Such copolymers, in which the monomers are present in a weight ratio of about 20:80, are available commercially as an approx. 50% non-aqueous polymer dispersion under the name Salcare® SC 92.

Further preferred cationic polymers are for example
quaternized cellulose derivatives such as are available commercially under the names Celquat® and Polymer JR®. The compounds Celquat® H 100, Celquat® L 200 and Polymer JR® 400 are preferred quaternized cellulose derivatives,
cationic alkyl polyglycosides according to DE-PS 44 13 686,
cationized honey, for example the commercial product Honeyquat® 50,
cationic guar derivatives, such as in particular the products sold under the trade names Cosmedia® Guar and Jaguar®,
polysiloxanes containing quaternary groups, such as for example the commercially available products Q2-7224 (manufacturer: Dow Corning; a stabilized trimethylsilyl amodimethicone), Dow Corning® 929 Emulsion (containing a hydroxyl-amino-modified silicone, which is also known as amodimethicone), SM-2059 (manufacturer: General Electric), SLM-55067 (manufacturer: Wacker) as well as Abil®-Quat 3270 and 3272 (manufacturer: Th. Goldschmidt), diquaternary polydimethylsiloxanes, Quaternium-80,
polymeric dimethyl diallyl ammonium salts and copolymers thereof with esters and amides of acrylic acid and methacrylic acid. The products available commercially under the names Merquat® 100 (poly(dimethyl diallyl ammonium chloride)) and Merquat® 550 (dimethyl diallyl ammonium chloride acrylamide copolymer) are examples of such cationic polymers,
quaternized polyvinyl alcohol,
and the polymers known under the names Polyquaternium-2, Polyquaternium-17, Polyquaternium-18 and Polyquaternium-27 with quaternary nitrogen atoms in the polymer main chain.

Further cationic polymers that can be used according to the invention are the “temporary cationic” polymers. These polymers conventionally contain an amino group that at certain pH values takes the form of a quaternary ammonium group and is therefore cationic. Chitosan and derivatives thereof, such as are widely available commercially under the trade names Hydagen® CMF, Flydagen® HCMF, Kytamer® PC and Chitolam® NB/101, for example, are preferred.

The agents according to the invention preferably contain the conditioning, cationic and/or amphoteric polymers in an amount from 0.01 to 5 wt. %, in particular in an amount from 0.1 to 2 wt. %, relative in each case to the total application preparation.

Further preferred agents according to the invention are characterized in that they additionally contain conditioning substance(s)—relative to their weight—in amounts from 0.001 to 10 wt. %, preferably 0.005 to 7.5 wt. %, more preferably 0.01 to 5 wt. % and in particular 0.05 to 2.5 wt. %, with preferred conditioning substance(s) being selected from the group comprising L-carnitine and/or salts thereof; panthenol and/or pantothenic acid; 2-furanones and/or derivatives thereof (in particular pantolactone); taurine and/or salts thereof; niacinamide; ubiquinone; ectoine; allantoin.

L-Carnitine (IUPAC name (R)-(3-Carboxy-2-hydroxypropyl)-N,N,N-trimethylammonium hydroxide), is a naturally occurring, vitamin-like substance. L-Carnitine derivatives that are preferred according to the invention are selected in particular from acetyl L-carnitine, L-carnitine fumarate, L-carnitine citrate, lauroyl L-carnitine and more preferably L-carnitine tartrate. The specified L-carnitine compounds are available for example from Lonza GmbH (Wuppertal, Germany). Preferred agents according to the invention are characterized in that they contain—relative to their weight—0.001 to 10 wt. %, preferably 0.005 to 7.5 wt. %, more preferably 0.01 to 5 wt. % and in particular 0.05 to 2.5 wt. % of L-carnitine or L-carnitine derivatives, preferred L-carnitine derivatives being selected from acetyl L-carnitine, L-carnitine fumarate, L-carnitine citrate, lauroyl L-carnitine and in particular L-carnitine tartrate.

Panthenol (IUPAC name: (+)-(R)-2,4-Dihydroxy-N-(3-hydroxypropyl)-3,3-dimethylbutyramide) is converted in the body to pantothenic acid. Pantothenic acid is a vitamin from the group of B vitamins (vitamin B5). Preferred agents according to the invention are characterized in that they contain—relative to their weight—0.01 to 5 wt. %, preferably 0.05 to 2.5 wt. %, more preferably 0.1 to 1.5 wt. % and in particular 0.25 to 1 wt. % panthenol ((±)-2,4-dihydroxy-N-(3-hydroxypropyl)-3,3-dimethylbutyramide).

A further conditioning enhancer that can preferably be used, which has activating properties, is taurine. Agents that are preferred according to the invention contain—relative to their weight—0.01 to 15 wt. %, preferably 0.025 to 12.5 wt. %, more preferably 0.05 to 10 wt. %, more preferably 0.1 to 7.5 wt. % and in particular 0.5 to 5 wt. % of taurine (2-aminoethanesulfonic acid).

A further preferred group of conditioning enhancers in the agents according to the invention are vitamins, provitamins or vitamin precursors. These are described below:

In summary, agents according to the invention are preferred which contain—relative to their weight—0.1 to 5 wt. %, preferably 0.2 to 4 wt. %, more preferably 0.25 to 3.5 wt. %, more preferably 0.5 to 3 wt. % and in particular 0.5 to 2.5 wt. % of vitamins and/or provitamins and/or vitamin precursors, which are preferably assigned to groups A, B, C, E, F and H, wherein preferred agents contain-2,4-dihydroxy-N-(3-hydroxypropyl)-3,3-dimethylbutyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or folic acid (vitamin B₉, vitamin Bₑ or vitamin M) and/or vitamin B₆ and/or vitamin B₁₂.

It has been found that certain quinones are particularly suitable as conditioning enhancers. More preferred agents according to the invention are characterized in that as a conditioning substance they contain—relative to their weight—0.0001 to 1 wt. %, preferably 0.001 to 0.5 wt. % and more preferably 0.005 to 0.1 wt. % of at least one ubiquinone and/or at least one ubiquinol and/or at least one derivative of these substances, wherein preferred agents contain a ubiquinone of formula (Ubi)

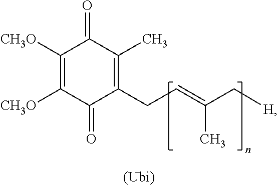

in which n denotes the values 6, 7, 8, 9 or 10, more preferably 10 (coenzyme Q10).

As a further conditioning enhancer the agents according to the invention can contain ectoine. Ectoine ((4S)-2-methyl-1,4,5,6-tetrahydropyrimidine-4-carboxylic acid) is a natural substance belonging to the group of compatible solutes.

As a further constituent the agents according to the invention can contain at least one carbohydrate from the group of monosaccharides, disaccharides and/or oligosaccharides. Hair treatment agents that are preferred according to the invention are characterized in that as a conditioning substance they contain—relative to their weight—0.01 to 5 wt. %, preferably 0.05 to 4.5 wt. %, more preferably 0.1 to 4 wt. %, more preferably 0.5 to 3.5 wt. % and in particular 0.75 to 2.5 wt. % of carbohydrate(s), selected from monosaccharides, disaccharides and/or oligosaccharides, wherein preferred carbohydrates are selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose
disaccharides, in particular sucrose and/or maltose and/or lactose and/or trehalose and/or cellobiose and/or gentiobiose and/or isomaltose.

In a further preferred embodiment the agents according to the invention can contain emulsifiers (F). Emulsifiers cause water-stable or oil-stable adsorption layers, which protect the dispersed droplets against coalescence and thus stabilize the emulsion, to form at the phase interface. Like surfactants, emulsifiers are therefore made up of a hydrophobic and a hydrophilic molecule part. Hydrophilic emulsifiers preferably form O/W emulsions and hydrophobic emulsifiers preferably form W/O emulsions. An emulsion is understood to be a distribution in droplet form (dispersion) of one liquid in another liquid, expending energy to create stabilizing phase interfaces by means of surfactants. The choice of these emulsifying surfactants or emulsifiers is governed by the substances to be dispersed and the external phase in each case as well as by the fine-particle character of the emulsion. Emulsifiers that can be used according to the invention are for example
addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group,
C₁₂-C₂₂ fatty acid monoesters and diesters of addition products of 1 to 30 mol of ethylene oxide with polyols having 3 to 6 carbon atoms, in particular with glycerol, ethylene oxide and polyglycerol addition products with methyl glucoside fatty acid esters, fatty acid alkanol amides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as the sugar component are preferred,
mixtures of alkyl (oligo)glucosides and fatty alcohols, for example the commercially available product Montanov® 68,
addition products of 5 to 60 mol of ethylene oxide with castor oil and hydrogenated castor oil,
partial esters of polyols having 3 to 6 carbon atoms with saturated fatty acids having 8 to 22 C atoms,
sterols. Sterols are understood to be a group of steroids which bear a hydroxyl group on C atom 3 of the steroid skeleton and are isolated from both animal tissue (zoosterols) and vegetable fats (phytosterols). Examples of zoosterols are cholesterol and lanosterol. Examples of suitable phytosterols are ergosterol, stigmasterol and sitosterol. Sterols known as mycosterols are also isolated from fungi and yeasts,
phospholipids. These are understood above all to be the glucose phospholipids which are obtained for example as lecithins or phosphatidyl cholines from for example egg yolk or plant seeds (e.g. soybeans),
fatty acid esters of sugars and sugar alcohols such as sorbitol,
polyglycerols and polyglycerol derivatives such as for example polyglycerol poly-12-hydroxystearate (commercial product Dehymuls® PGPH),
linear and branched fatty acids having 8 to 30 C atoms and Na, K, ammonium, Ca, Mg and Zn salts thereof.

The agents according to the invention contain the emulsifiers preferably in amounts from 0.1 to 25 wt. %, in particular 0.5 to 15 wt. %, relative to the total agent. The compositions according to the invention can preferably contain at least one non-ionogenic emulsifier having an HLB value of 8 to 18. Non-ionogenic emulsifiers having an HLB value of 10 to 15 can be more preferred according to the invention.

Depending on the nature of the agent according to the invention, it may be necessary for it also to contain at least one surfactant. This applies in particular in the case of skin cleaning agents and shampoos. However, other agents, such as for example hair rinses, hair masks and certain styling agents, in particular styling foams, can also contain surfactants.

Cationic surfactants can be used for example, as already described above as suitable conditioning substances. With regard to the preferred cationic surfactants and the amounts used, the details given above apply correspondingly.

In addition to or in place of the cationic surfactants, the agents can contain further surfactants or emulsifiers, with both anionic and ampholytic and non-ionic surfactants and all types of known emulsifiers being suitable in principle. The group of ampholytic or amphoteric surfactants comprises zwitterionic surfactants and ampholytes. The surfactants may already have an emulsifying action.

All anionic surface-active substances which are suitable for use on the human body are suitable in principle as anionic surfactants. These are characterized by a water-solubilizing anionic group such as for example a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxyl sulfonates,
sulfated hydroxyalkyl polyethylene and/or hydroxyalkylene propylene glycol ethers,
sulfonates of unsaturated fatty acids having 8 to 24 C atoms and 1 to 6 double bonds,
esters of tartaric acid and citric acid with alcohols that are addition products of around 2 to 15 molecules of ethylene oxide and/or propylene oxide with fatty alcohols having 8 to 22 C atoms,
alkyl and/or alkenyl ether phosphates,
sulfated fatty acid alkylene glycol esters of formula (E1-II)

R⁷CO(AlkO)ₙSO₃M (E1-11)
in which R⁷CO denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a cation such as are described in DE-OS 197 36 906,
amide ether carboxylic acids,
condensation products of C₈ to C₃₀ fatty alcohols with protein hydrolysates and/or amino acids and derivatives thereof, which are known to the person skilled in the art as protein fatty acid condensates, such as for example the Lamepon® types, Gluadin® types, Hostapon® KCG or Amisoft® types.

Preferred anionic surfactants are alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and protein fatty acid condensates.

Surface-active compounds classed as zwitterionic surfactants are those bearing at least one quaternary ammonium group and at least one —COO⁽⁻⁾ or —SO₃⁽⁻⁾ group in the molecule. Particularly suitable zwitterionic surfactants, are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are understood to be surface-active compounds which in addition to a C₈-C₂₄ alkyl or acyl group contain at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. More preferred ampholytes are N-cocoalkyl aminopropionate, cocoacylaminoethyl aminopropionate and C₁₂-C₁₈ acyl sarcosine.

Non-ionic surfactants contain as a hydrophilic group a polyol group, a polyalkylene glycol ether group or a combination of a polyol and polyglycol ether group, for example. Such compounds are for example
addition products of 2 to 50 mol of ethylene oxide and/or 1 to 5 mol of propylene oxide with linear and branched fatty alcohols having 8 to 30 C atoms, with fatty acids having 8 to 30 C atoms and with alkyl phenols having 8 to 15 C atoms in the alkyl group,
addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydrol® LS, Dehydrol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol types (Cognis),
alkoxylated triglycerides,
alkoxylated fatty acid alkyl esters of formula (E4-I)

R¹CO—(OCH₂CHR²)_{w}OR³ (E4-I)
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type according to formula (E4-II),

R⁴O-[G]ₚ (E4-II)
in which R⁴ denotes an alkyl or alkenyl residue having 4 to 22 carbon atoms, G denotes a sugar residue having 5 or 6 carbon atoms and p denotes numbers from 1 to 10. They can be obtained by means of the relevant methods of preparative organic chemistry. The preferred alkyl and/or alkenyl oligoglycosides are thus alkyl and/or alkenyl oligoglucosides. The index value p in the general formula (E4-II) indicates the degree of oligomerization (DP), i.e. the distribution of mono- and oligoglycosides, and denotes a number between 1 and 10. The alkyl or alkenyl residue R⁴ can derive from primary alcohols having 4 to 11, preferably 8 to 10 carbon atoms. Alkyl oligoglucosides based on hydrogenated C_{12/14} coconut alcohol with a DP of 1 to 3 are preferred.

The alkylene oxide addition products with saturated linear fatty alcohols and fatty acids each containing 2 to 30 mol of ethylene oxide per mol of fatty alcohol or fatty acid have proved themselves as preferred non-ionic surfactants. Preparations having outstanding properties are likewise obtained if they contain fatty acid esters of ethoxylated glycerol as non-ionic surfactants. These compounds are characterized by the following parameters. The alkyl residue R contains 6 to 22 carbon atoms and can be both linear and branched. Primary linear aliphatic residues and aliphatic residues that are methyl-branched in the 2-position are preferred. Such alkyl residues are for example 1-octyl, 1-decyl, 1-lauryl, 1-myristyl, 1-cetyl and 1-stearyl. 1-Octyl, 1-decyl, 1-lauryl and 1-myristyl are more preferred. If oxo alcohols are used as starting materials, compounds having an odd number of carbon atoms in the alkyl chain dominate.

The compounds having alkyl groups which are used as the surfactant can each be uniform substances. It is generally preferable, however, to use native vegetable or animal raw materials as starting materials for these substances, such that mixtures of substances having differing alkyl chain lengths depending on the individual raw material are obtained. The additional surfactants are generally used in amounts from 0.1 to 45 wt. %, preferably 0.5 to 30 wt. % and particularly preferably from 0.5 to 25 wt. %, relative in each case to the total composition. The amount used depends substantially on the purpose of the agent according to the invention. If it is a shampoo or another cleaning agent, surfactant amounts above 45 wt. % are also usual.

The agents according to the invention can be formulated in all conventional forms for cosmetic agents, for example in the form of solutions, which can be applied to the skin or hair as a face or hair lotion or a pump or aerosol spray, in the form of creams, emulsions, waxes, gels or surfactant-containing foaming solutions or other preparations that are suitable for application on the skin or hair.

The compositions according to the invention are very suitable in particular for stabilizing gas bubbles in the agent. Air or other gases or gas mixtures can be readily incorporated into the agents according to the invention in this way with long-term stability. This can optionally take place at the time the agents are produced, by pressurizing the agent with gas, preferably air, before filling, such that a product containing visible gas bubbles is filled.

In such a procedure, preferred agents according to the invention are obtained which contain gas bubbles, preferably air bubbles. Preferred cosmetic agents are characterized in that the agent is in the form of a foam, which preferably has a density below 0.9 g/cm³, preferably between 0.3 and 0.8 g/cm³ and in particular between 0.5 and 0.7 g/cm³. The agents according to the invention in foam form are characterized by an unexpected foam stability.

The production of foamed agents can take place in a variety of ways. The pressurization of the agents with the gas required for foaming preferably takes place in combination with a mixing process in which at least two, preferably at least half and preferably all of the ingredients contained in the agent are mixed together. Examples of such mixing processes are
Extrusion processes in which the mixture of substances to be extruded is pressurized with gas in the extruder;
Extrusion processes in which the mixture of substances to be extruded is charged with an expanding agent in the extruder;
Mixing processes in which gas, preferably air, is incorporated into the mixture of substances at the same time as the mixing rotors are acting. In this variant the incorporation of the gas is preferably brought about by the movement of the mixing rotors. Such a procedure is known as “whipping”.

The foamed or whipped agents are then filled into the containers intended for transportation and/or sale.

The present application therefore also provides a method for producing a cosmetic agent according to the invention, in which an optionally premixed blend of the ingredients of the cosmetic agent is mixed, characterized in that the density of the resulting cosmetic agent is at least 10%, preferably at least 20%, more preferably 20 to 70% and in particular 30 to 60% below the density of the optionally premixed blend employed in the method.

Such methods for producing a cosmetic agent according to the invention are preferred in particular in which an optionally premixed blend of the ingredients of the cosmetic agent is mixed by the action of mechanical mixing rotors in such a way that the density of the resulting cosmetic agent is at least 10%, preferably at least 20%, more preferably 20 to 70% and in particular 30 to 60% below the density of the optionally premixed blend employed in the method.

Oil-in-water emulsions are produced to particular advantage by means of this method.

It is however also possible and preferable to provide application devices in which the unpressurized agent is pressurized with gas, preferably air, on application. The present invention therefore also provides a kit-of-parts comprising an agent according to the invention and an application device that pressurizes the agent with a gas, preferably with air, before it is dispensed.

The invention therefore secondly provides a method for the temporary shaping of keratinic fibers in which the cosmetic agent according to the invention is pressurized with a gas and then applied to the hair.

All that has been stated in respect of the agents according to the invention applies with necessary alterations to the method according to the invention. The desired shaping of the hair can take place with the fingers or hands or with suitable, conventional aids such as a comb or brush for example.

The invention thirdly provides the use of the agents according to the invention for the temporary shaping of keratinic fibers. The agents according to the invention and products containing said agents are characterized in particular in that they impart a very strong styling hold to the treated hair without making the hair brittle or inflexible. Instead a pleasant, soft touch is achieved.

The formulation of the agents according to the invention can take place in all conventional forms for cosmetic agents. The composition of a number of more preferred cosmetic agents can be taken from the table below (amounts in wt. % relative to the total weight of the cosmetic agent unless otherwise specified).

| | Formula 1 | Formula 2 | Formula 3 | Formula 4 | Formula 5 |
|---|---|---|---|---|---|
| Monoester* | 0.1 to 15 | 0.3 to 10 | 0.5 to 3 | 1.0 | 2.8 |
| Diester* | 0.1 to 15 | 0.3 to 10 | 0.5 to 3 | 1.0 | 1.0 |
| Film-forming polymer | 0.2 to 15 | 0.4 to 10 | 0.75 to 5.0 | 0.5 | 2.3 |
| Wax (mp 40° C. to 90° C.) | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density [g/cm³] | 0.3 to 0.8 | 0.3 to 0.8 | 0.5 to 0.7 | 0.60 | 0.74 |

*Mono- or diesters of optionally alkylated sugars with C₆-C₃₀ fatty acids

| | | | | | Formula |
|---|---|---|---|---|---|
| | Formula 6 | Formula 7 | Formula 8 | Formula 9 | 10 |
| Sucrose monostearate | 0.1 to 15 | 0.3 to 10 | 0.5 to 3 | 1.0 | 2.8 |
| Sucrose distearate | 0.1 to 15 | 0.3 to 10 | 0.5 to 3 | 1.0 | 1.0 |
| Film-forming polymer | 0.2 to 15 | 0.4 to 10 | 0.75 to 5.0 | 0.5 | 2.3 |
| Wax (mp 40° C. to 90° C.) | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density [g/cm³] | 0.3 to 0.8 | 0.3 to 0.8 | 0.5 to 0.7 | 0.60 | 0.74 |

| | Formula | Formula | Formula | Formula | Formula |
|---|---|---|---|---|---|
| | 11 | 12 | 13 | 14 | 15 |
| Monoester* | 0.1 to 15 | 0.3 to 10 | 0.5 to 3 | 1.0 | 2.8 |
| Diester* | 0.1 to 15 | 0.3 to 10 | 0.5 to 3 | 1.0 | 1.0 |
| Modified starch | 0.2 to 15 | 0.4 to 10 | 0.75 to 5.0 | 0.5 | 2.3 |
| Wax (mp 40° C. to 90° C.) | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density [g/cm³] | 0.3 to 0.8 | 0.3 to 0.8 | 0.5 to 0.7 | 0.60 | 0.74 |

*Mono- or diesters of optionally alkylated sugars with C₆-C₃₀ fatty acids

| | Formula | Formula | Formula | Formula | Formula |
|---|---|---|---|---|---|
| | 16 | 17 | 18 | 19 | 20 |
| Monoester* | 0.1 to 15 | 0.3 to 10 | 0.5 to 3 | 1.0 | 2.8 |
| Diester* | 0.1 to 15 | 0.3 to 10 | 0.5 to 3 | 1.0 | 1.0 |
| Film-forming polymer | 0.2 to 15 | 0.4 to 10 | 0.75 to 5.0 | 0.5 | 2.3 |
| Beeswax | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density [g/cm³] | 0.3 to 0.8 | 0.3 to 0.8 | 0.5 to 0.7 | 0.60 | 0.74 |

*Mono- or diesters of optionally alkylated sugars with C₆-C₃₀ fatty acids

| | Formula | Formula | Formula | Formula | Formula |
|---|---|---|---|---|---|
| | 21 | 22 | 23 | 24 | 25 |
| Monoester* | 0.1 to 15 | 0.3 to 10 | 0.5 to 3 | 1.0 | 2.8 |
| Diester* | 0.1 to 15 | 0.3 to 10 | 0.5 to 3 | 1.0 | 1.0 |
| Film-forming polymer | 0.2 to 15 | 0.4 to 10 | 0.75 to 5.0 | 0.5 | 2.3 |
| Fatty acid (mp 40° C. to | 1.0 to 45 | 3.0 to 30 | 4.0 to 25 | 7.0 | 18 |
| 90° C.) | | | | | |
| Water | 40 to 95 | 50 to 90 | 60 to 85 | 70 | 67 |
| Misc | to 100 | to 100 | to 100 | to 100 | to 100 |
| Density [g/cm³] | 0.3 to 0.8 | 0.3 to 0.8 | 0.5 to 0.7 | 0.60 | 0.74 |

*Mono- or diesters of optionally alkylated sugars with C₆-C₃₀ fatty acids

| | Formu- | Formu- | Formu- | Formu- | Formu- |
|---|---|---|---|---|---|
| | la 26 | la 27 | la 28 | la 29 | la 30 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Wax | 1.0 to 45 | 3.0 to 30 | 4.0 to 25 | 7.0 | 18 |
| (mp 40° C. | | | | | |
| to 90° C.) | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 31 | la 32 | la 33 | la 34 | la 35 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Beeswax | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 36 | la 37 | la 38 | la 39 | la 40 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Fatty acid | 1.0 to 45 | 3.0 to 30 | 4.0 to 25 | 7.0 | 18 |
| (mp 40° C. | | | | | |
| to 90° C.) | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 41 | la 42 | la 43 | la 44 | la 45 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Beeswax | 1.0 to 20 | 2.0 to 15 | 4.0 to 10 | 8.0 | 12 |
| Fatty acid | 1.0 to 20 | 2.0 to 15 | 4.0 to 10 | 8.0 | 4.0 |
| (mp 40° C. | | | | | |
| to 90° C.) | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 46 | la 47 | la 48 | la 49 | la 50 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Beeswax | 1.0 to 45 | 3.0 to 30 | 4.0 to 25 | 7.0 | 18 |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 51 | la 52 | la 53 | la 54 | la 55 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Fatty acid | 1.0 to 45 | 3.0 to 30 | 4.0 to 25 | 7.0 | 18 |
| (mp 40° C. | | | | | |
| to 90° C.) | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 56 | la 57 | la 58 | la 59 | la 60 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Beeswax | 1.0 to 20 | 2.0 to 15 | 4.0 to 10 | 8.0 | 12 |
| Fatty acid | 1.0 to 20 | 2.0 to 15 | 4.0 to 10 | 8.0 | 4.0 |
| (mp 40° C. | | | | | |
| to 90° C.) | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

| | Formu- | Formu- | Formu- | Formu- | Formu- |
|---|---|---|---|---|---|
| | la 61 | la 62 | la 63 | la 64 | la 65 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Wax | 1.0 to 45 | 3.0 to 30 | 4.0 to 25 | 7.0 | 18 |
| (mp 40° C. | | | | | |
| to 90° C.) | | | | | |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 66 | la 67 | la 68 | la 69 | la 70 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Beeswax | 1.0 to 45 | 3.0 to 30 | 4.0 to 25 | 7.0 | 18 |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 71 | la 72 | la 73 | la 74 | la 75 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Fatty acid | 1.0 to 45 | 3.0 to 30 | 4.0 to 25 | 7.0 | 18 |
| (mp 40° C. | | | | | |
| to 90° C.) | | | | | |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 76 | la 77 | la 78 | la 79 | la 80 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Modified | 0.2 to 15 | 0.4 to 10 | 0.75 to 5.0 | 0.5 | 2.3 |
| starch | | | | | |
| Beeswax | 1.0 to 20 | 2.0 to 15 | 4.0 to 10 | 8.0 | 12 |
| Fatty acid | 1.0 to 20 | 2.0 to 15 | 4.0 to 10 | 8.0 | 4.0 |
| (mp 40° C. | | | | | |
| to 90° C.) | | | | | |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 81 | la 82 | la 83 | la 84 | la 85 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Beeswax | 1.0 to 45 | 3.0 to 30 | 4.0 to 25 | 7.0 | 18 |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 86 | la 87 | la 88 | la 89 | la 90 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Fatty acid | 1.0 to 45 | 3.0 to 30 | 4.0 to 25 | 7.0 | 18 |
| (mp 40° C. | | | | | |
| to 90° C.) | | | | | |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

| | Formu- | Formu- | Formu- | Formu- | Formu- |
|---|---|---|---|---|---|
| | la 91 | la 92 | la 93 | la 94 | la 95 |
| Sucrose | 0.1 to 15 | 0.3 to 10 | 0.5 to 3 | 1.0 | 2.8 |
| monostearate | | | | | |
| Sucrose | 0.1 to 15 | 0.3 to 10 | 0.5 to 3 | 1.0 | 1.0 |
| distearate | | | | | |
| Film-forming | 0.2 to 15 | 0.4 to 10 | 0.75 to 5.0 | 0.5 | 2.3 |
| polymer | | | | | |
| Beeswax | 1.0 to 20 | 2.0 to 15 | 4.0 to 10 | 8.0 | 12 |
| Fatty acid | 1.0 to 20 | 2.0 to 15 | 4.0 to 10 | 8.0 | 4.0 |
| (mp 40° C. | | | | | |
| to 90° C.) | | | | | |
| Organic | 0.1 to 15 | 0.1 to 15 | 1.0 to 10 | 5.0 | 8.0 |
| solvent ** | | | | | |
| Water | 40 to 95 | 50 to 90 | 60 to 85 | 70 | 67 |
| Misc | to 100 | to 100 | to 100 | to 100 | to 100 |
| Density | 0.3 to 0.8 | 0.3 to 0.8 | 0.5 to 0.7 | 0.60 | 0.74 |
| [g/cm³] | | | | | |

** Preferably glycerol

### EXAMPLES

Unless otherwise specified, the quantities given below are percentages by weight.

The styling agents E1 to E4 according to the invention were produced in accordance with Table 1 below.

TABLE 1

| Raw material (INCI name) | E1 | E2 | E3 | E4 |
|---|---|---|---|---|
| Sucrose monostearate | 1.0 | 1.5 | 1.0 | 1.5 |
| Sucrose distearate | 1.0 | 1.0 | 1.5 | 1.5 |
| Polyvinylpyrrolidone | 1.5 | 1.75 | 2.0 | 1.25 |
| Beeswax | 5.0 | 5.5 | 4.0 | 4.5 |
| Stearic acid | 7.5 | 7.75 | 8.0 | 7.5 |
| Palmitic acid | 7.35 | 7.5 | 7.0 | 7.0 |
| Glycerol | 8.0 | 8.0 | 8.0 | 8.0 |
| Propanediol-1,2 | 1.0 | 1.0 | 1.0 | 1.0 |
| Phenoxyethanol | 0.5 | 0.5 | 0.5 | 0.5 |
| Dye | 0.002 | 0.002 | 0.002 | 0.002 |
| Perfume | 0.10 | 0.10 | 0.10 | 0.20 |
| Water, deionized | to 100 | to 100 | to 100 | to 100 |

The agents were produced by conventional mixing of the raw materials listed in the table and used for hair styling by rubbing onto the palms of the hands and then applying to dry or wet hair.

The hair exhibited a soft shine, outstanding styling hold and excellent remodeling ability.

The interesting texture of agents E1 to E4 also made it possible to incorporate air bubbles into the agents during the production thereof. The air bubbles had diameters of 0.5 to 2 mm and were stable even when the products were stored for several weeks.

In a further series of experiments the compositions E1 to E4 according to the invention were introduced into an application device which incorporates air into the gel-like product on application. Products containing air bubbles were able to be applied, in which the diameter of the air bubbles was 0.5 to 2 mm.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for the temporary shaping of keratinic fibers, comprising in a cosmetically acceptable carrier, relative in each case to the total agent:
a) 0.1 to 15 wt. % of monoesters of optionally alkylated sugars with C₆-C₃₀ fatty acids,
b) 0.1 to 15 wt. % of diesters of optionally alkylated sugars with C₆-C₃₀ fatty acids,
c) 0 to 50 wt. % of at least one film-forming polymer, and
d) 0 to 50 wt. % of at least one wax with a melting point in a range from 40° C. to 90° C.,
with the proviso that the proportion of component(s) c) and d) in the cosmetic agent is 0.2 to 50 wt. %.

2. The cosmetic agent according to claim 1, characterized in that component a) is selected from the group consisting of sucrose monostearate, sucrose monococoate, methyl glucoside monostearate, ethyl glucoside monolaurate, ethyl glucoside monococoate and butyl glucoside monococoate.

3. The cosmetic agent according to claim 1, characterized in that component b) is selected from the group consisting of sucrose distearate, sucrose dicocoate, methyl glucoside isostearate, ethyl glucoside dilaurate and ethyl glucoside dicocoate.

4. The cosmetic agent according to claim 1, characterized in that the agent comprises 0.2 to 12 wt. % monoesters of optionally alkylated sugars with C₆-C₃₀ fatty acids.

5. The cosmetic agent according to claim 1, characterized in that the agent comprises 0.2 to 12 wt. % diesters of optionally alkylated sugars with C₆-C₃₀ fatty acids.

6. The cosmetic agent according to claim 1, characterized in that the agent comprises 0.25 to 5 wt. % sucrose monostearate and 0.25 to 5 wt. % sucrose distearate.

7. The cosmetic agent according to claim 1, characterized in that the agent comprises 1 to 45 wt. % at least one wax from the group consisting of beeswax, carnauba wax, candelilla wax, montan wax and cetyl palmitate.

8. The cosmetic agent according to claim 1, characterized in that the agent comprises 0.2 to 15 wt. % film-forming polymer(s).

9. The cosmetic agent according to claim 1, characterized in that the agent comprises 1 to 45 wt. % beeswax and 0.2 to 15 wt. % film-forming polymer(s).

10. The cosmetic agent according to claim 1, characterized in that the weight ratio of the wax included in the agent to the esters of optionally alkylated sugars with C₆-C₃₀ fatty acids included in the agent is >8:1.

11. The cosmetic agent according to claim 1, characterized in that the agent is in the form of a foam, which has a density below 0.9 g/cm³.

12. A method for producing a cosmetic agent according to claim 1, comprising:
mixing an optionally premixed blend of the ingredients of the cosmetic agent, characterized in that the density of the resulting cosmetic agent is at least 10% below the density of the blend.

13. A kit-of-parts, comprising an agent according to claim 1 and an application device that pressurizes the agent with a gas before it is dispensed.

14. A method for the temporary shaping of hair, comprising:
pressurizing a cosmetic agent according to claim 1 with a gas, and
applying the agent to the hair.
